(19) United States
(12) Patent Application Publication
(10) Pub. No.: US 2021/0236260 A1
(43) Pub. Date: Aug. 5, 2021

(54) STENT-GRAFT

(71) Applicant: Board of Regents of the University of Nebraska, Lincoln, NE (US)

(72) Inventors: Jason N. MacTaggart, Omaha, NE (US); Alexey Kamenskiy, Omaha, NE (US); Kaspars Maleckis, Omaha, NE (US); Anastasia Desyatova, Omaha, NE (US)

(21) Appl. No.: 17/051,097

(22) PCT Filed: Apr. 30, 2019
(86) PCT No.: PCT/US2019/030041

§ 371 Date: Oct. 27, 2020

Related U.S. Application Data
(60) Provisional application No. 62/664,424, filed on Apr. 30, 2018.

(51) Int. Cl. A61F 2/07; A61L 27/04

(52) U.S. Cl. CPC A61F 2/07; A61L 27/047

(57) ABSTRACT

A stent-graft apparatus includes a membrane configured to exhibit one or more mechanical properties in a range corresponding to a range for the one or more mechanical properties for human vascular tissue, and a scaffold coupled to the membrane, the scaffold including one or more struts.

### CROSS-REFERENCE TO RELATED APPLICATIONS

This application claims priority under 35 U.S.C. § 119 to U.S. Provisional Application Ser. No. 62/664,424, filed on Apr. 30, 2018, which is incorporated by reference in its entirety herein.

### TECHNICAL FIELD

This disclosure relates to apparatus and methods for vascular stent grafting.

### BACKGROUND

Large elastic arteries, such as the aorta, possess a Windkessel effect. The Windkessel effect in the aorta is the result of the aorta distending following ejection of blood from the left ventricle (LV) during systole, and recoiling during diastole. The Windkessel effect of the aorta assists in moving blood distally to the periphery during cardiac relaxation and in perfusing the coronary bed. In addition, the Windkessel effect helps control peak blood pressure and flow waves by providing a cushioning effect that protects the heart and downstream organs from pressure injury. Increases in aortic stiffness diminish the Windkessel effect, overwork the heart, and cause abnormal blood pressure and blood flow distally. Increases in aortic stiffness may eventually lead to pathological changes to the heart, brain, and kidneys.

### SUMMARY

In an example implementation, a stent-graft apparatus includes a membrane configured to exhibit one or more mechanical properties in a range corresponding to a range for the one or more mechanical properties for human vascular tissue, and a scaffold coupled to the membrane, the scaffold including one or more struts.

In an aspect combinable with the example implementation, the one or more mechanical properties includes a Windkessel effect.

In another aspect combinable with any of the previous aspects, the membrane is configured to exhibit deformation in a range of 10% to 20% in response to pressure in a range of 40 mmHg to 150 mmHg.

In another aspect combinable with any of the previous aspects, the membrane is configured to exhibit less than 25% deformation in response to pressures in a range of 40 mmHg to 150 mmHg.

In another aspect combinable with any of the previous aspects, the scaffold is configured to exert a pressure less than a maximum pressure against an adjacent blood vessel following implantation of the stent-graft into a lumen of the blood vessel.

In another aspect combinable with any of the previous aspects, pressures above the maximum pressure result in increased stress in the blood vessel.

In another aspect combinable with any of the previous aspects, the membrane includes at least one of a woven material, a non-woven material, or a fiberless material.

In another aspect combinable with any of the previous aspects, the membrane includes a nanofiber-based material.

In another aspect combinable with any of the previous aspects, an orientation of one or more nanofibers of the nanofiber-based material provides at least one of the one or more mechanical properties in the range corresponding to the range for the one or more mechanical properties for human vascular tissue.

In another aspect combinable with any of the previous aspects, the membrane includes a material configured to exhibit nonlinear stretch behavior.

In another aspect combinable with any of the previous aspects, the material is configured to exhibit a greater ability to stretch when subjected to pressures in a range of 40 mmHg to 120 mmHg than when subjected to pressures greater than 120 mmHg.

In another aspect combinable with any of the previous aspects, the membrane includes a material configured to exhibit anisotropic stretch behavior.

In another aspect combinable with any of the previous aspects, the membrane includes a polymer.

In another aspect combinable with any of the previous aspects, the membrane includes at least one of polymethacrylate, poly vinyl phenol, polyvinylchloride, polyvinyl alcohol, polyacrylamide, poly(lactic-co-glycolic) acid (PLGA), collagen, polycaprolactone (PCL), polyurethane, Pellethane® thermoplastic polyurethane 2363-55DE, Pellethane® thermoplastic polyurethane 2363-55D, Pellethane® thermoplastic polyurethane 5863-82A, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene glycol, poly(ethylene-co-vinyl alcohol), polyacrylnitrile, polyvinyl pyrrolidone, polymetha-phenylene isophthalamide, gelatin, alginate, chitosan, starch, pectin, cellulose, methylcellulose, sodium polyacrylate, resilin, or starch-acrylonitrile co-polymers.

In another aspect combinable with any of the previous aspects, membrane is manufactured by at least one of electrospinning, electrospraying, electroblowing, melt spinning, wet spinning, film casting, film blowing, weaving, knitting, extrusion, solution casting, or spin casting methods.

In another aspect combinable with any of the previous aspects, the scaffold includes at least one of nitinol, magnesium, a magnesium alloy, biodegradable plastic, non-biodegradable plastic, metal, a metal alloy, or a polymer.

In another aspect combinable with any of the previous aspects, the scaffold includes a biodegradable material.

In another aspect combinable with any of the previous aspects, the scaffold includes at least one of biodegradable magnesium or a biodegradable magnesium alloy.

In another aspect combinable with any of the previous aspects, a thickness of the one or more struts is in a range of 200 μm to 1000 μm.

In another aspect combinable with any of the previous aspects, an amplitude of the one or more struts is in a range of 2 mm to 6 mm.

Potential benefits of the one or more implementations described in the present specification may include improved patient outcomes following stent-graft implantation. The one or more implementations may provide patient improved blood flow and blood pressure following stent-graft implantation. The one or more implementations may reduce the risk of left ventricular remodeling and mass increase following stent-graft implantation. The one or more implementations may reduce the risk of injury to peripheral organs due to high blood pressure or abnormal blood flow. The one or more implementations may also reduce or prevent arterial stiffening of the aorta and/or peripheral arteries following stent-graft implantation. The one or more implementations may also help restore the artery elasticity, such as the elasticity of an aorta. The one or more implementations may accommodate natural growth of the aorta with age. The one or more implementations may reduce the likelihood of endoleaks. The one or more implementations may provide improved biological function to cells of the aorta and facilitate endothelialization. The one or more implementations may reduce the risk of aortic dissection.

It is appreciated that methods in accordance with the present disclosure may include any combination of the aspects and features described herein. That is, methods in accordance with the present disclosure are not limited to the combinations of aspects and features specifically described herein, but also include any combination of the aspects and features provided.

The details of one or more implementations of the subject matter of this disclosure are set forth in the accompanying drawings and the description below. Other features, objects, and advantages of the subject matter will be apparent from the description and drawings, and from the claims.

### DESCRIPTION OF DRAWINGS

FIG. 1 depicts mechanical properties of various stent-graft fabrics, human aortas, and porcine aortas.

FIGS. 2A-2B show perspective views of a stent-graft, in accordance with some embodiments.

FIG. 2C shows a top view of a stent-graft, in accordance with some embodiments.

FIGS. 3A and 3B depict the stretching ability of a material for a membrane of a stent-graft, in accordance with some embodiments.

FIG. 4A depicts nonlinear stretch behavior of a material for a membrane of a stent-graft, in accordance with some embodiments.

FIG. 4B depicts anisotropic stretch behavior of a material for a membrane of a stent-graft, in accordance with some embodiments.

FIGS. 5A and 5B depict the mechanical properties of a PET-based stent-graft and a nanofiber-based stent-graft, respectively, in response to physiological flow.

FIG. 6 depicts the permeability of a nanofiber-based membrane material of a stent-graft, in accordance with some embodiments.

FIG. 7 depicts the strength of a nanofiber-based membrane material of a stent-graft and the strength of a human aorta.

FIG. 8 depicts the cell coverage of a nanofiber-based material and an ePTFE-based material seeded with porcine aortic vascular smooth muscle cells at 7 days in culture.

FIGS. 9A and 9B depict biocompatibility of a nanofiber-based material and an ePTFE material, respectively.

FIG. 10 depicts various designs for a scaffold of a stent-graft, in accordance with some embodiments.

FIG. 11 depicts a parametric analysis of various scaffold designs for a stent-graft.

FIG. 12 depicts an example stent-graft, in accordance with some embodiments.

FIG. 13 depicts an example scaffold for a stent-graft, in accordance with some embodiments.

### DETAILED DESCRIPTION

Implementations of the present disclosure are directed to a stent-graft apparatus that exhibits mechanical properties within a range of the mechanical properties of healthy human vascular tissue. More particularly, implementations of the present disclosure are directed to a stent-graft that exhibits the Windkessel effect under pressure.

Stent-grafting is a first-line therapy for most aortic pathologies and trauma. Traditionally, aortic stent-grafting is conducted by installing a stiff, rigid stent-graft into a portion of the aorta of a patient. For example, current aortic stent-grafts are typically made with expanded polytetrafluoroethylene (ePTFE) or polyethylene terephthalate (PET) materials that are substantially stiffer than the aortic wall. While stiff stent-grafts are generally thought to work well in the aorta, the long-term effects of stiff aortic stent-grafts associated with aortic stiffening have not been highlighted in stent-graft literature, which focuses primarily on mortality and local complications requiring re-intervention.

Studies have recently been conducted that demonstrate that implantation of stiff ePTFE or PET stent-grafts into the aorta results in loss of the natural Windkessel effect of the aorta. The loss of the natural Windkessel effect following ePTFE/PET stent-graft implantation overworks the heart, producing an increase in left ventricular mass, and causes abnormal blood pressure and blood flow distally, which can eventually lead to pathology of the left ventricle, brain, and kidneys.

While the Windkessel effect is most strong in young healthy aortas, weak hearts of the older patients with degenerative aortic disease may be more susceptible to the loss of the remaining Windkessel effect. Therefore, both younger and older patients may benefit from a stent-graft device that would offload the heart by producing some of the Windkessel functions.

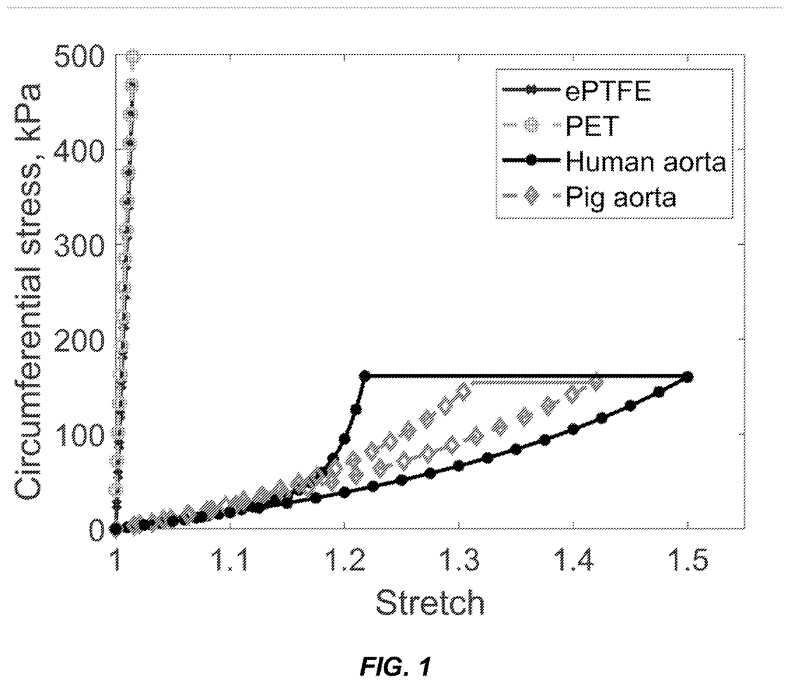

FIG. 1 depicts results of a recent study comparing the mechanical properties of conventional ePTFE and PET stent-graft fabrics with the mechanical properties of a human aorta and a porcine aorta. As depicted in FIG. 1, the ePTFE and PET stent-grafts exhibited substantially reduced stretch, and, thus, were substantially stiffer, than either the human or porcine aorta. Due to this increase in stiffness, traditional PTFE and PET stent-grafts fail to produce a Windkessel effect. In addition, due to the difference in stiffness between ePTFE and PET stent-grafts and human aortas, implantation of traditional PTFE and PET stent-grafts in the aorta results in local, artificial stiffening of the aorta at the implantation site, resulting in a reduction of the Windkessel effect produced by the aorta.

Another recent study demonstrated that implantation of a standard stiff thoracic aortic stent-graft in young trauma patients resulted in a 19% to 45% increase in the mass of the left ventricle and a 16% to 30% increase in left ventricle wall thickness. Similar outcomes were reported in elderly patients with abdominal aortic stent-grafts.

Porcine studies were conducted to determine the effects of implantation of conventional stiff PTFE/PET stent-grafts in the thoracic descending aorta on blood flow and blood pressure. The porcine studies demonstrated that implantation of the convention ePTFE/PET stent-grafts in the thoracic descending aorta significantly affected pressure waveforms in the aorta, the carotid, coronary, and renal arteries, producing higher systolic pressure, and resulting in 2.5-fold increase in pulse wave velocity, which is a known marker of cardiovascular pathology.

In view of the above context, implementations of the present disclosure are directed to a stent-graft that exhibits mechanical properties within the same range of the mechanical properties of healthy human vascular tissue. For example, implementations of the subject matter of the disclosure are directed to a Windkessel-preserving (or aortic elasticity preserving) stent-graft that reduces artificial stiffening at the implantation, and promotes normal blood pressure and blood flow.

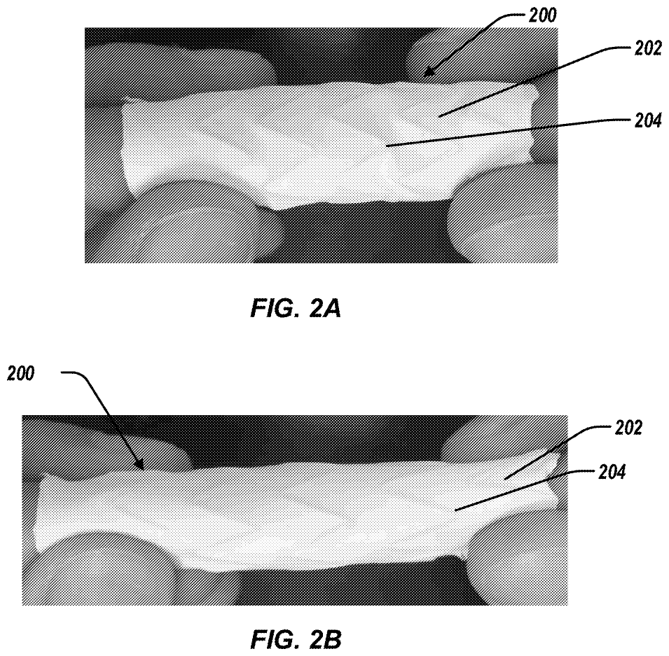

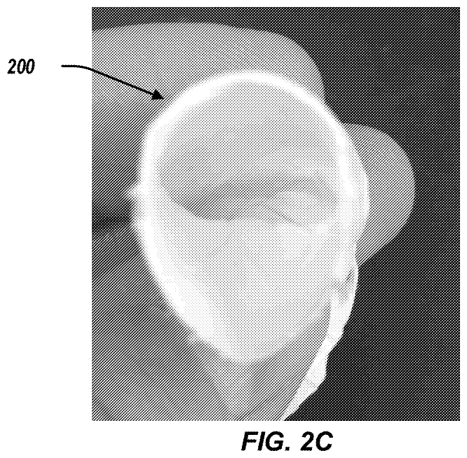

FIGS. 2A-2C depict an example stent-graft 200 in accordance with implementations of the present disclosure. As depicted in FIGS. 2A-2B, the stent-graft 200 includes a membrane 202 and a scaffold 204. The scaffold 204 is embedded in the membrane 202 to provide structure and stability to the stent-graft 200. In some implementations, scaffold 204 can be attached to the exterior surface or interior surface of the stent-graft 200 using sutures, glue, or other methods. FIG. 2A depicts the stent-graft 200 in an unstretched state, and FIG. 2B depicts the stent-graft 200 in a stretched state. As depicted in FIGS. 2A and 2B, the stent-graft is compliant and can be stretched significantly, similar to the stretching capabilities (i.e., compliance) of a human aorta.

FIG. 2C depicts a top view of an example stent-graft 200 such that the interior of the stent-graft 200 is visible. As depicted in FIG. 2C, the stent-graft 200 has an open-ended tubular shape.

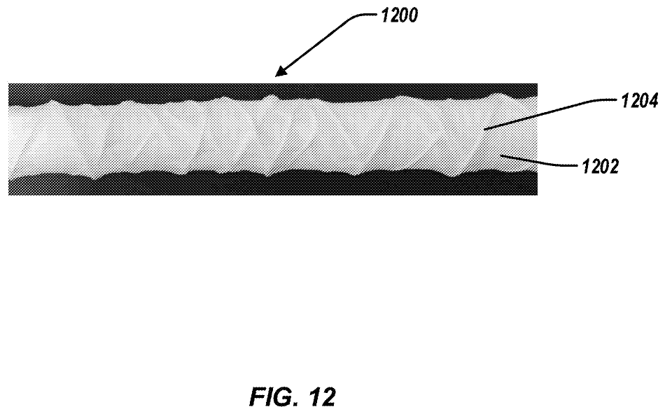

FIG. 12 depicts another example stent-graft 1200 with an alternate scaffold 1204 design. As depicted in FIG. 12, example stent-graft 1200 includes a membrane 1202 and a scaffold 1204.

As described in further detail herein, the manufacture and design of the stent-graft 200 can be adjusted to provide improved mechanical properties of the stent-graft 200 that closely mimic the mechanical properties of healthy vascular tissues. In some examples, stent-grafts 200 may be used to restore the mechanical properties of an artery, such as an aorta, that has lost elasticity due to disease or other causes.

The stent-graft 200 may be used in the treatment of various vascular diseases and pathologies, including, but not limited to aortic diseases and pathologies. For example, the stent-graft 200 can be used to the treat aortic aneurysms (e.g., abdominal aortic aneurysms and thoracic aortic aneurysms), aortic dissections, aortic stenosis, traumatic injury to the aorta, coarctation, and genetic defects.

In some implementations, the membrane 202 of the stent-graft 200 is composed of a non-woven material. In some examples, the membrane 202 of the stent-graft 200 is composed of a woven material. In some implementations, the membrane 202 material may be composed of submicron fibers, microfibers, porous fiberless films, or a combination thereof.

In some implementations, the membrane 202 of the stent-graft 200 is composed of one or more polymers. For example, membrane 202 may include one or more pure, functionalized, or modified polymers including, but not limited to, polymethacrylate, poly vinyl phenol, polyvinylchloride, polyvinyl alcohol, polyacrylamide, poly(lactic-co-glycolic) acid (PLGA), collagen, polycaprolactone (PCL), polyurethanes, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene glycol, poly(ethylene-co-vinyl alcohol), polyacrylonitrile, polyvinyl pyrrolidone, polymetha-phenylene isophthalamide, gelatin, alginate, chitosan, starch, pectin, cellulose, methylcellulose, sodium polyacrylate, resilin, and starch-acrylonitrile co-polymers.

In some examples, the membrane 202 material is composed of a polyurethane-based polymer. In some implementations, one or more polymers are used to form the membrane 202. For example, the membrane 202 may be composed of one or more commercially-available polyurethanes from Lubrizol. In some examples, membrane 202 is composed of one or more Pellethane® thermoplastic polyurethanes including, but not limited, to 2363-55DE, 2363-55D, and 5863-82A. In some implementations, the polymers 2363-55DE and 5683-82A are used to form the membrane 202. Various ratios of different polymers may be used to form the membrane 202. In one embodiment, the polymers 2363-55DE and 5683-82A are combined in a ratio of 80% 5683-82A and 20% 2363-55DE to form the membrane 202 of the stent-graft 200. In some examples, membrane 202 is composed of a combination of polyurethane and nylon. In some implementations, membrane 202 is composed of a combination of polyurethane and PET. In some implementations, the membrane 202 is composed of an elastomeric material.

Membrane 202 can be manufactured using a variety of methods. Methods for manufacturing membrane 202 can include, but are not limited to, electrospinning, electrospraying, electroblowing, melt spinning, wet spinning, film casting, film blowing, weaving, knitting, extrusion, solution casting, and spin casting methods. The fibers of the membrane 202 may be woven. As previously discussed, in some implementations, the material used for the membrane 202 may be fiberless. Methods of manufacturing a fiberless membrane 202 material can include, but are not limited to, extrusion, solution casting, or spin casting.

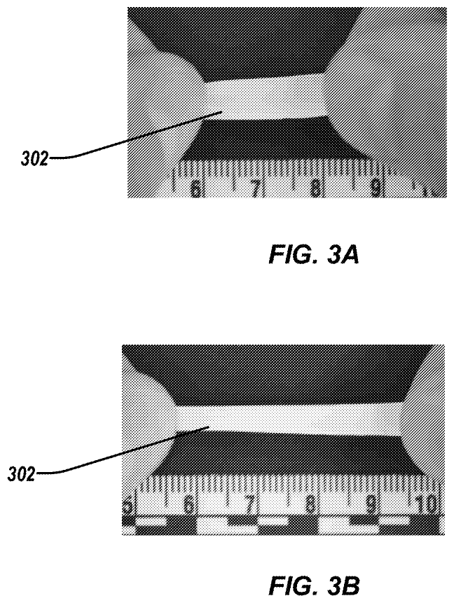

In some implementations, the membrane 202 is designed with mechanical properties, such as compliance and/or elasticity, in the same range as the mechanical properties of human vascular tissue. In some examples, the membrane 202 can be designed to exhibit mechanical properties in the same range as the mechanical properties of a healthy human aorta. For example, the membrane 202 can be designed with a compliant, elastic membrane material. FIGS. 3A and 3B depict an example material 302 for a membrane 202 of a stent-graft 200. FIG. 3A shows the membrane material 302 in an unstretched state without any load applied to the ends of the material 302. FIG. 3B depicts the membrane material 302 in a stretched state after applying a force to each end of the material 302. As depicted in FIGS. 3A and 3B, the material 302 exhibits an ability to stretch along its longitudinal axis (i.e., the material 302 exhibits compliance along its longitudinal axis). The elasticity and compliance of the membrane material 302 may be optimized to be in the same range as the elasticity and compliance of a human aorta.

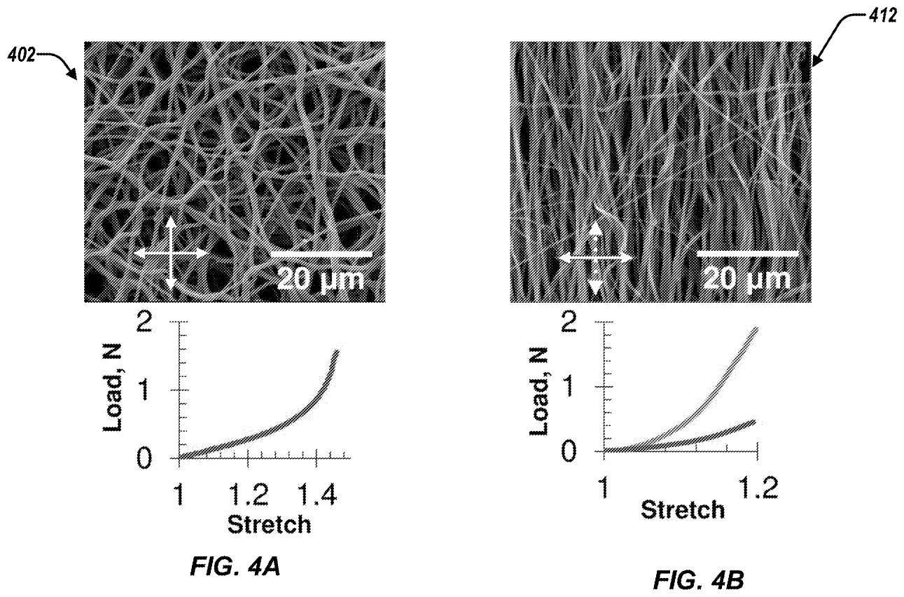

In some implementations, the membrane 202 is a nanofiber-based membrane 202. The mechanical properties of nanofiber-based membrane 202 can be optimized by adjusting the orientation, size, or composition of the fibers of the nanofiber membrane 202 material. For example, FIGS. 4A and 4B depict two nanofiber membrane materials 402, 412. Nanofiber membrane material 402 and nanofiber membrane material 412 are composed of the same polymer. However, as depicted in FIGS. 4A and 4B, the orientation of the nanofibers differs between the two materials 402, 412. The orientation of the nanofibers of material 402 is random, while the nanofibers of material 412 are highly oriented. As depicted in FIGS. 4A and 4B, membrane 402 exhibited greater ability to stretch (i.e., greater compliance) along its longitudinal axis than membrane 412 under the same load. Also, membrane 412 exhibited a different ability to stretch (i.e., compliance) in the longitudinal direction and the circumferential direction, while membrane 402 exhibited the same ability to stretch (i.e., compliance) in both the longitudinal and circumferential directions, as depicted in FIGS. 4A and 4B. These differences in compliance between the membrane materials 402, 412 are the result of the differences in orientation of the nanofibers of each of the materials 402, 412.

In some implementations, the membranes 202 are composed of electrospun nanofibers. By using different electrospinning processes to manufacture the membrane 202 materials, membranes 202 can be produced with different orientations of nanofibers. As previously discussed, changing the orientation of the nanofibers of the membrane 202 material changes the mechanical properties of the membrane 202, such as the compliance of the membrane 202. Therefore, by producing membrane 202 material using different electrospinning manufacturing process, membrane 202 materials having different and optimized mechanical properties can be created.

In some examples, the membrane 202 of the stent-graft 200 is composed of a nonlinear material. Tensile testing of a non-linear membrane 202 material was conducted, and the results of the tensile testing are depicted in FIG. 4A. As depicted in FIG. 4A, the nanofiber membrane 202 material exhibited nonlinear tensile behavior, as the load required to stretch the material an incremental amount increased nonlinearly above a stretch of 1.2. Nonlinear membrane 202 materials provide the stent-graft 200 with mechanical characteristics that are similar to the mechanical characteristics of human vascular tissue. For example, the stent-graft may include a membrane 202 that exhibits the same nonlinear stretch characteristics (i.e., same nonlinear compliance) as vascular tissue.

In some implementations, the membrane 202 deforms 10% to 20% in response to applied pressures in a normal physiological pressure range. In some implementations, the membrane 202 deforms 10% to 20% when subjected to pressures in a range of 40 mmHg to 150 mmHg. In one example embodiment, a membrane 202 having a circumference of 100 mm when subjected to a pressure below 40 mmHg may to deform (i.e., stretch) to provide a circumference of 110 mm when subjected to pressures in a range of 40 mmHg to 150 mmHg. In some examples, a membrane 202 having a circumference of 100 mm when subjected to a pressure below 40 mmHg may to deform (i.e., stretch) to provide a circumference that ranges from 110 mm to 120 mm when subjected to pressures in a range of 40 mmHg to 150 mmHg. In one example embodiment, a membrane 202 with a length of 50 mm when subjected to a pressure below 40 mmHg may to deform (i.e., stretch) to a length of 60 mm when subjected to pressures in a range of 40 mmHg to 150 mmHg. In some examples, a membrane 202 with a length of 50 mm when subjected to a pressure below 40 mmHg may to deform (i.e., stretch) to a length that ranges from 55 mm to 60 mm when subjected to pressures in a range of 40 mmHg to 150 mmHg. In some embodiments, the membrane 202 deforms 10% to 20% when subjected to pressures in a range of 40 mmHg to 120 mmHg. In some implementations, the membrane 202 deforms 10% to 20% when subjected to pressures in a range of 40 mmHg to 200 mmHg.

In some examples, the membrane 202 material deforms 25% or less when subjected to pressures in a normal physiological pressure range. In some examples, the membrane 202 material deforms 20% or less when subjected to pressures in a normal physiological pressure range. In some implementations, the membrane 202 material deforms 25% or less when subjected to pressures in a range of 40 mmHg to 150 mmHg. In one example embodiment, a membrane 202 having a circumference of 100 mm when subjected to a pressure below 40 mmHg may be configured to deform (i.e., stretch to a circumference no larger than 125 mm when subjected to pressures in a range of 40 mmHg to 150 mmHg. In one example embodiment, a membrane 202 having a length of 50 mm when subjected to a pressure below 40 mmHg may be configured to deform (i.e., stretch) to a length no larger than 62.5 mm when subjected to pressures in a range of 40 mmHg to 150 mmHg. In some implementations, the membrane 202 material deforms 25% or less when subjected to pressures in a range of 40 mmHg to 120 mmHg. In some examples, the membrane 202 material deforms 20% or less when subjected to pressures in a range of 40 mmHg to 120 mmHg. In some examples, the membrane 202 material deforms 25% or less when subjected to pressures in a range of 40 mmHg to 200 mmHg. In some examples, the membrane 202 material deforms 20% or less when subjected to pressures in a range of 40 mmHg to 200 mmHg.

In some implementations, the deformation of the membrane 202 when subjected to pressures in a range of 40 mmHg to 150 mmHg is elastic deformation. In some examples, the deformation of the membrane 202 when subjected to pressures in a range of 40 mmHg to 120 mmHg is elastic deformation. In some examples, the deformation of the membrane 202 when subjected to pressures in a range of 40 mmHg to 200 mmHg is elastic deformation.

By using membrane 202 material that exhibits nonlinear stretch properties (i.e., nonlinear compliance), the mechanical behavior membrane 202 of the stent-graft 200 more closely mimics the mechanical behavior of vascular tissue than a membrane made of material having linear stretch behavior. For example, nonlinear membrane 202 allows for stretching when subjected to pressures between 40 mmHg and 120 mmHg, which helps cushion flow and provide the Windkessel effect to a blood flow (i.e., the membrane 202 exhibits a first level of compliance when subjected to pressures under 120 mmHg). In addition, membranes 202 composed of nonlinear material may resist stretching at applied pressures greater than 120 mmHg (i.e., membranes 202 may exhibit a second, reduced level of compliance when subjected to pressures greater than 120 mmHg), which helps prevent over bulging of the membrane 202 when the stent-graft is subjected to high blood pressures in vivo. For example, membrane 202 may exhibit increased ability to stretch (i.e., increased compliance) when subjected to pressures in a range of 40 mmHg to 120 mmHg, and exhibit reduced ability to stretch (i.e., reduced compliance) when subjected to pressures greater than 120 mmHg. In some examples, membrane 202 may exhibit increased ability to stretch (i.e., increased compliance) when subjected to pressures in a range of 40 mmHg to 150 mmHg, and exhibit reduced ability to stretch (i.e., reduced compliance) when subjected to pressures greater than 150 mmHg. In some examples, membrane 202 may exhibit increased ability to stretch (i.e., increased compliance) when subjected to pressures in a range of 40 mmHg to 200 mmHg, and exhibit reduced ability to stretch (i.e., reduced compliance) when subjected to pressures over 200 mmHg.

In some examples, the nonlinear behavior of the membrane 202 can be optimized to correspond to the nonlinear mechanical behavior of vascular tissue, such as the nonlinear behavior of the human aorta. In some examples, the nonlinear mechanical behavior of the membrane 202 can be optimized by adjusting the arrangement and size of fibers of the membrane 202 material. For example, non-linear stretching behavior (i.e., compliance) of the membrane 202 can be adjusted by altering the shape of the fibers, such as by making the fibers of the membrane 202 wavy or undulating. In some implementations, increasing the undulations of the fibers of the membrane 202 results in a membrane 202 with increased nonlinear stretching behavior (i.e., increased nonlinear compliance). In some implementations, nonlinearity of the membrane 202 can be controlled and adjusted by creating a complex stress state in the fibers of the membrane. In some examples, creating a complex stress state in the fibers of the membrane 202 to control nonlinear membrane behavior is accomplished using thermal, mechanical, or hygroscopic techniques.

In some examples, different materials with different properties can be combined to create a membrane 202 with optimized nonlinear stretch behavior. For example, fibers with differing stiffnesses can be combined to provide a membrane 202 with optimized nonlinear stretch (i.e., compliance) behavior and provide an optimized stretch response to high pressures. For example, stiffer, undulating fibers can be combined with softer fibers to optimize membrane 202 stiffness and stretch in response to pressures greater than 120 mmHg.

In some examples, the membrane 202 of the stent-graft 200 is an anisotropic material. Tensile testing of an anisotropic membrane 202 material was conducted, and the results of the tensile testing are depicted in FIG. 4B. As depicted in FIG. 4B, the nanofiber membrane 202 material exhibited anisotropic behavior, as the load required to stretch the material an incremental amount in a first direction was greater than the load required to stretch the material an incremental amount in a second direction.

Anisotropic membrane 202 materials provide the stent-graft 200 with mechanical characteristics similar to the mechanical characteristics of human vascular tissue, as many vascular tissues exhibit anisotropic behavior. For example, the aorta may exhibit different ability to stretch (i.e., compliance) in the longitudinal direction and the circumferential direction. By using a membrane 202 material that exhibits anisotropic mechanical behavior, the compliance of the membrane 202 of the stent-graft 200 can be designed to exhibit the anisotropic mechanical behavior of the blood vessel in which the stent-graft 200 will be implanted. For example, an anisotropic membrane 202 can be used to design a stent-graft 200 with a first compliance in the longitudinal direction and a second, different compliance in the circumferential direction. In some examples, the membrane 202 can be designed to exhibit greater compliance in the longitudinal direction than in the circumferential direction.

In some examples, the anisotropic mechanical behavior of the membrane 202 can be optimized to correspond to the same anisotropic mechanical behavior of vascular tissue, such as the anisotropic mechanical behavior of the aorta. In some examples, the anisotropic mechanical behavior of the membrane 202 can be optimized by adjusting the arrangement and size of nanofibers of the membrane 202 material. As previously discussed, the orientation of the nanofibers of material 402 in FIG. 4A is random, while the nanofibers of material 412 in FIG. 4B are highly oriented. The material 402 of FIG. 4A demonstrated an isotropic mechanical response (i.e., mechanical properties that are similar in two directions). In contrast, material 412 of FIG. 4B exhibited anisotropic mechanical behavior as a result of the alignment of the fibers of material 412.

Anisotropy of the membrane 202 material may be characterized by the ratio of mechanical properties in two directions. For example, the anisotropy of the membrane 202 may be characterized based on the ratio of the amount of stretch of the membrane 202 in the longitudinal direction to the amount of stretch of the membrane 202 in the circumferential direction when the membrane 202 is subjected to a pressure. In some examples, the anisotropy of the membrane 202 as defined by the ratio of the amount of stretch of the membrane 202 in the longitudinal direction to the amount of stretch of the membrane 202 in the circumferential direction in response to an applied pressure is in a range of 0.5 to 2. In some examples, the anisotropy of the membrane 202 as defined by the ratio of the amount of stretch of the membrane 202 in the longitudinal direction to the amount of stretch of the membrane 202 in the circumferential direction in response to an applied pressure is in a range of 1 to 2. In some examples, the anisotropy of the membrane 202 as defined by the ratio of the amount of stretch of the membrane 202 in the longitudinal direction to the amount of stretch in of the membrane 202 in the circumferential direction in response to an applied pressure is less than 5.

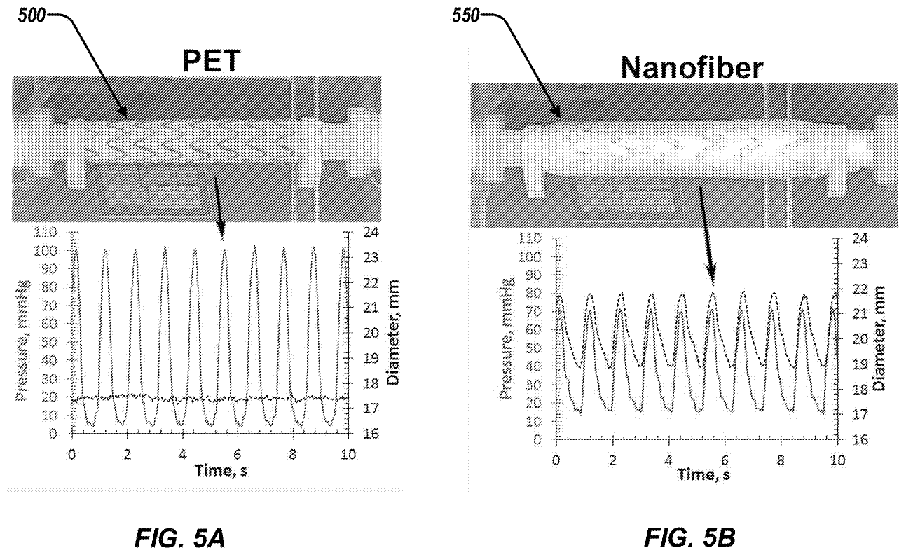

FIGS. 5A and 5B depict the results of performance testing of a stent-graft having a PET membrane 500 and of a stent-graft having a compliant nanofiber membrane 550, respectively. The performance of the stent-graft with a PET membrane 500 and the performance of the stent-graft with a compliant nanofiber membrane 550 were tested by implanting each of the stent-grafts 500, 550 in human aortas and attaching each of the implanted stent-grafts 500, 550 to a physiological flow circuit. The pressure produced by each of the stent-grafts 500, 550 and the changes in diameter of each of the stent-grafts 500, 550 were measured throughout the flow cycle. As shown in FIGS. 5A and 5B, the stent-graft with a nanofiber membrane 550 demonstrated lower systolic pressure for the same stroke volume compared to the PET-based stent-graft 500. Additionally, as depicted in FIGS. 5A and 5B, the nanofiber-based stent-graft 550 changed diameter during the cardiac flow cycle, while the stiff PET-based stent-graft 500 failed to change diameter. Based on the testing results depicted in FIGS. 5A and 5B, the stent-graft with the nanofiber membrane 550 demonstrated a significantly improved ability to maintain the Windkessel effect of the aorta compared to the stent-graft with a PET membrane 500. In addition, the stent-graft with a compliant nanofiber membrane 550 maintained normal pressure waveforms. In contrast, the stent-graft with the stiff PET membrane 500 failed to maintain the normal pressure wave forms of the human aorta, as depicted in FIG. 5A.

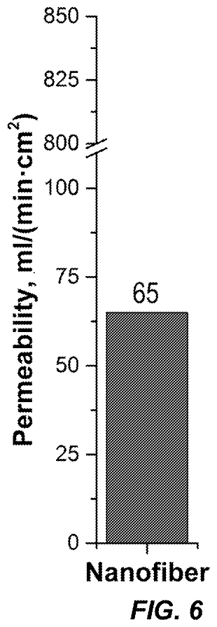

FIG. 6 depicts the results of the permeability testing of a nanofiber-based membrane for a stent-graft. Materials having a permeability over 800 ml/(min-cm²) require pre-clotting to provide an effective membrane that resists leaking. As can be seen in FIG. 6, the nanofiber membrane 202 demonstrated a permeability of 65 ml/(min-cm²). Based on these results, it was determined that a nanofiber-based membrane may be used in a stent-graft (such as stent-graft 200) without requiring pre-clotting. In some examples, generating a membrane 202 that does not require pre-clotting reduces the number of production steps required to manufacture the stent-graft 200.

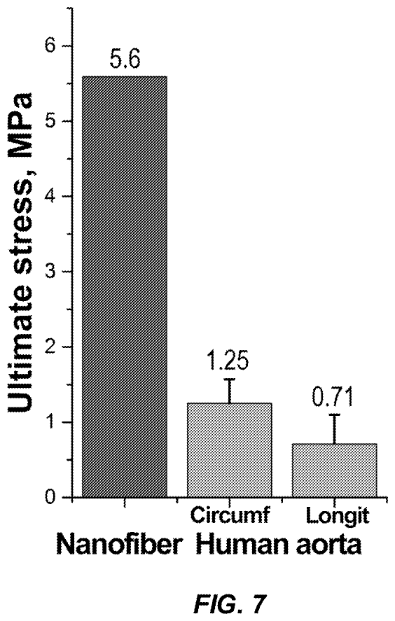

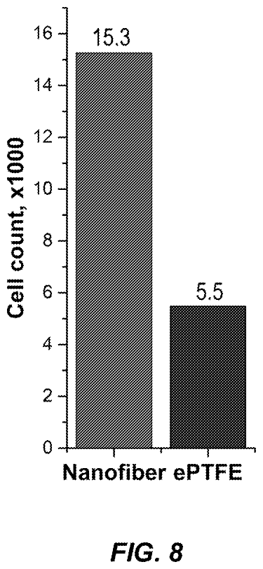

Nanofiber-based membrane 202 material was also subjected to tensile testing to determine the ultimate tensile strength of the membrane 202. The results of the tensile testing are depicted in FIG. 7. As can be seen in FIG. 7, the nanofiber-based membrane material exhibited a much higher ultimate tensile strength than the ultimate tensile strength of the human aorta in either the circumferential or longitudinal direction. These results demonstrate that the nanofiber-based membrane 202 material is significantly stronger than the human aorta. In some examples, the nanofiber-based membrane 202 is less susceptible to tearing or damage compared to ePTFE/PET membranes.

Additional studies were performed to determine the biocompatibility of nanofiber-based stent-graft membranes. In vitro cell viability and in vivo porcine studies were conducted using a nanofiber-based membrane 202 material. Based on these studies, it was determined that the nanofiber-based membrane material maintains its compliance in vivo, undergoes rapid endothelialization, and can be completely incorporated into the surrounding tissues. For example, the biocompatibility of a nanofiber-based membrane 202 material was compared with the biocompatibility of ePTFE by seeding samples of each material with porcine aortic vascular smooth muscle cells (vSMCs). The cell coverage of each material after 7 days of incubation is depicted in FIG. 8. As can be seen in FIG. 8, the nanofiber material exhibited better cell coverage and viability compared to the ePTFE.

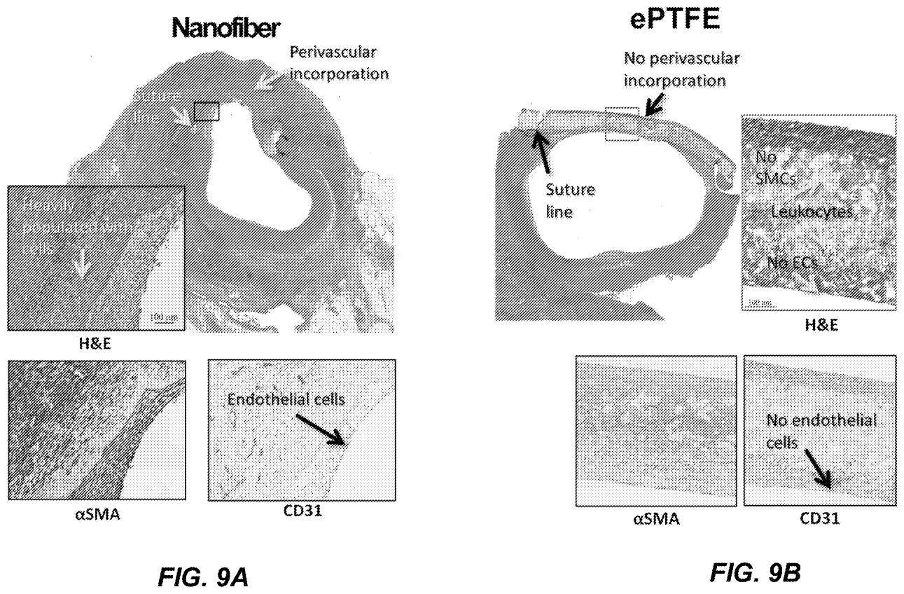

In order to further analyze the biocompatibility of the nanofiber-based material compared to ePTFE, a sample of nanofiber-based membrane material and a sample of ePTFE were each implanted in a swine iliac artery model. FIGS. 9A and 9B depict each artery model two weeks post-implantation. As can be seen in FIGS. 9A and 9B, the implanted nanofiber-based material experienced increased endothelialization compared to the ePTFE material. In addition, as depicted in FIGS. 9A and 9B, the nanofiber-based material exhibited perivascular incorporation, while the ePTFE material did not exhibit perivascular incorporation. These results indicate that nanofiber-based material is highly biocompatible, and exhibits improved biocompatibility over ePTFE, making the nanofiber-based material well suited for use as a stent-graft 200 membrane 202. In particular, it was determined that stent-grafts 200 with nanofiber-based membrane 202 can be more readily incorporated into the surrounding tissues following implantation compared to standard ePTFE stent-grafts.

Additionally, based on porcine studies using the nanofiber-based membrane 202, it was determined that use of a nanofiber-based membrane 202 in the stent-graft 200 can reduce the risk of endoleaks compared to stent grafting using standard ePTFE or PET stent-grafts. For example, as the membrane 202 becomes infiltrated with cells and integrated into the surrounding tissues, the cellular integration seals the fabric to the wall of the artery, which provides a biological fixation mechanism.

As previously discussed, stent-graft 200 also includes a scaffold 204. Scaffold 204 is embedded in the membrane 202 of the stent-graft 200 to provide structure and stability to the stent-graft 200. In some examples, the scaffold 204 can be attached to the inside of the stent-graft 200 or the outside of the stent-graft 200. The scaffold 204 can be designed to assist device stability and help preserve the mechanical properties of the blood vessel in which the stent-graft 200 is implanted. For example, scaffold 204 is designed to provide sufficient support to prevent the stent-graft 200 from collapsing, but does not apply excess pressure to the wall of the blood vessel in which the stent-graft 200 is implanted. By providing a scaffold 204 that applies a reduced amount of pressure to the blood vessel adjacent the stent-graft 200 compared to standard stent-graft scaffolds, the stent-graft 200 reduces interference with mechanical properties of the adjacent blood vessel compared to a standard PET/PTFE stent-graft, thus reducing trauma caused to the blood vessel. For instance, by including a scaffold 204 that minimizes pressure applied to the walls of the surrounding blood vessel, the stent-graft 200 better preserves and supports the Windkessel effect of the blood vessel (such as the aorta) post-implantation compared to standard, stiff PTFE or PET based stent-grafts.

In some examples, the scaffold 204 is configured to exert a pressure less than a maximum pressure against an adjacent blood vessel following implantation of the stent-graft 200 into a lumen of the blood vessel. In some examples, the scaffold 204 is configured to exert a pressure in a range of 0 Pascal to 6,000 Pascal against an adjacent blood vessel following implantation of the stent-graft 200 into a lumen of the blood vessel. In some examples, the scaffold 204 is configured to exert a pressure in a range of 0 Pascal to 5,000 Pascal against an adjacent blood vessel following implantation of the stent-graft 200 into a lumen of the blood vessel. In some examples, the scaffold 204 is configured to exert a pressure in a range of 0 Pascal to 3,000 Pascal against an adjacent blood vessel following implantation of the stent-graft 200 into a lumen of the blood vessel. In some examples, the scaffold 204 is configured to exert a pressure in a range of 0 Pascal to 1,000 Pascal against an adjacent blood vessel following implantation of the stent-graft 200 into a lumen of the blood vessel. In some examples, the scaffold 204 is configured to exert a pressure in a range of 0 Pascal to 800 Pascal against an adjacent blood vessel following implantation of the stent-graft 200 into a lumen of the blood vessel. In some examples, the scaffold 204 is configured to exert a pressure in a range of 0 Pascal to 500 Pascal against an adjacent blood vessel following implantation of the stent-graft 200 into a lumen of the blood vessel. In some examples, the scaffold 204 is configured to exert a pressure in a range of 0 Pascal to 250 Pascal against an adjacent blood vessel following implantation of the stent-graft 200 into a lumen of the blood vessel. In some examples, the scaffold 204 is configured to exert a pressure in a range of 0 Pascal to 100 Pascal against an adjacent blood vessel following implantation of the stent-graft 200 into a lumen of the blood vessel. In some examples, the scaffold 204 is configured to exert a pressure in a range of 0 Pascal to 50 Pascal against an adjacent blood vessel following implantation of the stent-graft 200 into a lumen of the blood vessel.

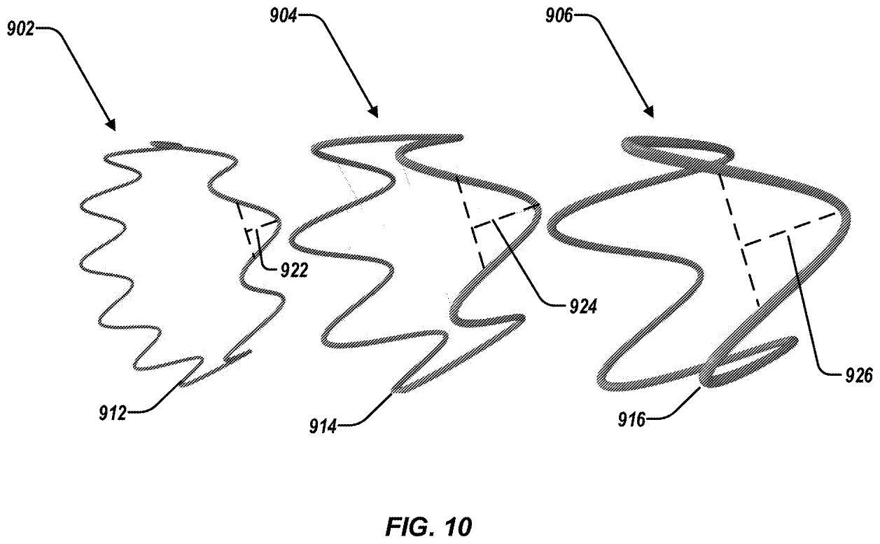

The design and pattern of the scaffold 204 of the stent-graft 200 may be adjusted to optimize the mechanical properties of the stent-graft 200. For example, the design and pattern of the scaffold 204 of the stent-graft 200 may be adjusted to optimize the pressure exerted by the scaffold 204 against an adjacent blood vessel following implantation of the stent-graft 200 into a lumen of the blood vessel. FIG. 10 depicts three example scaffold designs 902, 904, 906. As depicted in FIG. 10, the amplitude 922, 924, 926 of the undulations of the struts 912, 914, 916 of the scaffold 204 can be adjusted to optimize the mechanical properties of the scaffold 204. In some examples, the amplitude of the struts is the distance between the centerline of an undulation of struts to the peak of the undulation. In some examples, the amplitude of the struts is the distance between the centerline of an undulation of struts to the valley of the undulation. In some implementations, decreasing the amplitude 922, 924, 926 of the undulations of the struts 912, 914, 916 of the scaffold 204 increases the durability of scaffold 204. However, decreasing the amplitude 922, 924, 926 of the undulations of the struts 912, 914, 916 of the scaffold 204 also increases the stiffness of the scaffold 204, which results in increased pressure applied to the blood vessel by the scaffold 204. Application of increased pressure to a blood vessel by a stent-graft increases the risk of causing blood vessel injury or dissection.

For example, scaffold design 902 has struts 912 with a smaller amplitude 922 than the strut amplitudes 924, 926 of scaffold design 904 and 906, respectively. As a result, a scaffold 204 having design 902 exhibits better durability compared to scaffolds 204 with designs 904 and 906. However, as a result of the decreased amplitude 922 of the struts 912 of design 902 compared with the amplitudes 924, 926 of designs 904 and 906, stent-grafts 200 with a scaffold 204 having design 902 will also exert increased pressure on adjacent blood vessels compared to stent-grafts 200 with scaffolds 204 having designs 904 or 906, leading to increased stress in the blood vessel wall. In some examples, the amplitude of the struts of the scaffold 204 is in a range of 2 mm to 6 mm.

As shown in FIG. 10, the thickness of the struts of the scaffold 204 can also be adjusted to optimize the mechanical properties of the scaffold 204. In some examples, the thickness of the struts 912, 914, 916 is the diameter of a cross section of the one or more struts 912, 914, 916. In some examples, the thickness of the struts is a length of a side of a cross section of the one or more struts 912, 914, 916. In some implementations, increasing the thickness of the struts 912, 914, 916 of the scaffold 204 increases the durability and stability of the scaffold 204. However, increasing the thickness of the struts 912, 914, 916 of the scaffold 204 also increases the stiffness of the scaffold 204, which results in increased pressure applied to the blood vessel by the scaffold 204. As previously discussed, application of increased pressure to a blood vessel by a stent-graft increases the risk of blood vessel injury and dissection due to the stent-graft.

For example, scaffold design 906 has thicker struts 916 than scaffold design 902 or scaffold design 904, and thus a scaffold 204 having design 906 exhibits better durability compared to scaffolds 204 with designs 902 and 904. However, as a result of the increased strut 916 thickness, stent-grafts with scaffolds 204 having design 906 will also exert increased pressure on adjacent blood vessels compared to stent-grafts 200 with scaffolds 204 having design 902 or design 904, leading to increased stress in the blood vessel wall. In some examples, the thickness of the struts ranges from 200 μm to 1000 μm.

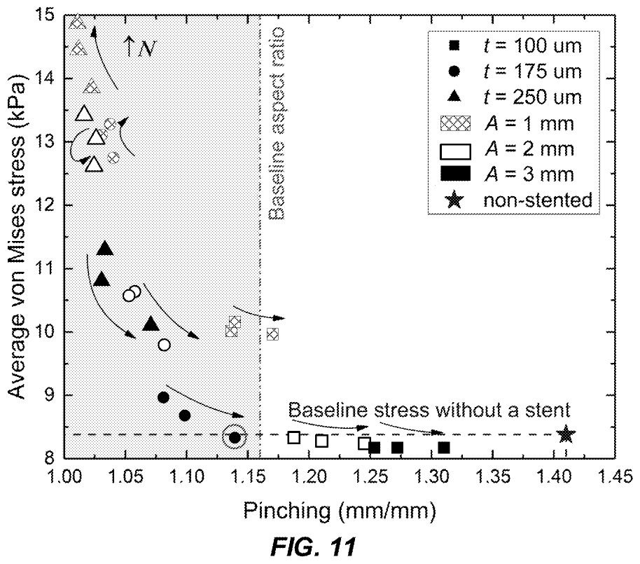

By adjusting both the amplitude 922, 924, 926 of the undulations of the struts 912, 914, 916 of the scaffold 204 and the thickness of the struts 912, 914, 916 of the scaffold 204, the scaffold 204 can be optimized to provide stability and exhibit good fatigue resistance without applying excess pressure to the blood vessel adjacent the stent-graft 200. A parametric analysis can be conducted to determine the optimum balance of scaffold 204 strut thickness and scaffold 204 strut amplitude. In some examples, parametric analysis can be conducted to determine the optimum scaffold design, including strut amplitude and thickness, for different categories of patients based on location in the aorta the stent-graft will be placed, the condition to be treated, patient age, patient gender, and risk factors of the patient. FIG. 11 depicts results of an example parametric analysis of the combined effects of scaffold strut thickness and scaffold strut amplitude on pinching of the scaffold, which is a measure of the ability of the scaffold to maintain its shape under load. As depicted in FIG. 11, the strut amplitude and the strut thickness of a stent-graft 200 scaffold 204 can be balanced to reduce stresses applied by the stent-graft and minimize stent-graft pinching.

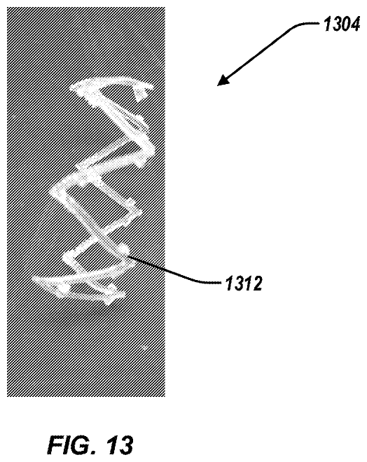

FIG. 13 depicts an example scaffold 1304. As shown in FIG. 13, the example scaffold 1304 includes a plurality of struts 1312 that connect together to form a series of peaks and valleys.

The embedded scaffold 204 of the stent-graft 200 may be composed of a number of materials including, but not limited to, nitinol, magnesium and magnesium alloys or mixtures (including biodegradable forms of magnesium), biodegradable plastic, non-biodegradable plastic, metal, metal alloys, polymers, and combinations of polymers. In some implementations, the embedded scaffold 204 is composed of nitinol. In some examples, the scaffold 204 is composed of a magnesium-based material. In some implementations, the scaffold 204 is composed of a biodegradable magnesium-based material. In some examples the scaffold 204 is composed of a polymer material.

The shape and design of the embedded scaffold 204 can take on a variety of forms to alter the mechanical characteristics of the stent-graft 200. In some implementations, the scaffold 204 has a pattern similar to the pattern of z-shape or wire-based stents. In some implementations, the scaffold 204 has a pattern different from the pattern of stents. As previously discussed with reference to FIG. 10, the design of the scaffold 204 may be altered by changing the thickness and amplitude of the struts of the scaffold. In addition, the size of the scaffold 204 may be adjusted based on the desired mechanical properties of the stent-graft 200. For example, the size of the scaffold 204 may be optimized based on the target blood vessel for implantation of the stent-graft 200. In some implementations, the shape of the scaffold 204 may be varied to provide one or more desired mechanical properties.

The embedded scaffold 204 may be manufactured using a variety of methods including, but not limited to, wire braiding, wire knitting, laser sheet cutting, laser tube cutting, 3D printing, machining, casting, or extrusion.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some examples be excised from the combination, and the claimed combination may be directed to a sub-combination or variation of a sub-combination.

A number of embodiments have been described. Nevertheless, it will be understood that various modifications may be made without departing from the spirit and scope of the disclosure. Accordingly, other embodiments are within the scope of the following claims.

What is claimed is:

1. A stent-graft apparatus comprising:
a membrane configured to exhibit one or more mechanical properties in a range corresponding to a range for the one or more mechanical properties for human vascular tissue; and
a scaffold coupled to the membrane, the scaffold comprising one or more struts.

2. The stent-graft of claim 1, wherein the one or more mechanical properties comprise a Windkessel effect.

3. The stent-graft of claim 1, wherein the membrane is configured to exhibit deformation in a range of 10% to 20% in response to pressure in a range of 40 mmHg to 150 mmHg.

4. The stent-graft of claim 1, wherein the membrane is configured to exhibit less than 25% deformation in response to pressures in a range of 40 mmHg to 150 mmHg.

5. The stent-graft of claim 1, wherein the scaffold is configured to exert a pressure less than a maximum pressure against an adjacent blood vessel following implantation of the stent-graft into a lumen of the blood vessel.

6. The stent-graft of claim 5, wherein pressures above the maximum pressure result in increased stress in the blood vessel.

7. The stent-graft of claim 1, wherein the membrane comprises at least one of a woven material, a non-woven material, or a fiberless material.

8. The stent-graft of claim 1, wherein the membrane comprises a nanofiber-based material.

9. The stent-graft of claim 8, wherein an orientation of one or more nanofibers of the nanofiber-based material provides at least one of the one or more mechanical properties in the range corresponding to the range for the one or more mechanical properties for human vascular tissue.

10. The stent-graft of claim 1, wherein the membrane comprises a material configured to exhibit nonlinear stretch behavior.

11. The stent-graft of claim 10, wherein the material is configured to exhibit a greater ability to stretch when subjected to pressures in a range of 40 mmHg to 120 mmHg than when subjected to pressures greater than 120 mmHg.

12. The stent-graft of claim 1, wherein the membrane comprises a material configured to exhibit anisotropic stretch behavior.

13. The stent-graft of claim 1, wherein the membrane comprises a polymer.

14. The stent-graft of claim 13, wherein the membrane comprises at least one of polymethacrylate, poly vinyl phenol, polyvinylchloride, polyvinyl alcohol, polyacrylamide, poly(lactic-co-glycolic) acid (PLGA), collagen, polycaprolactone (PCL), polyurethane, Pellethane® thermoplastic polyurethane 2363-55DE, Pellethane® thermoplastic polyurethane 2363-55D, Pellethane® thermoplastic polyurethane 5863-82A, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene glycol, poly(ethylene-co-vinyl alcohol), polyacrylnitrile, polyvinyl pyrrolidone, polymetha-phenylene isophthalamide, gelatin, alginate, chitosan, starch, pectin, cellulose, methylcellulose, sodium polyacrylate, resilin, or starch-acrylonitrile co-polymers.

15. The stent-graft of claim 1, wherein the membrane is manufactured by at least one of electrospinning, electrospraying, electroblowing, melt spinning, wet spinning, film casting, film blowing, weaving, knitting, extrusion, solution casting, or spin casting methods.

16. The stent-graft of claim 1, wherein the scaffold comprises at least one of nitinol, magnesium, a magnesium alloy, biodegradable plastic, non-biodegradable plastic, metal, a metal alloy, or a polymer.

17. The stent-graft of claim 1, wherein the scaffold comprises a biodegradable material.

18. The stent-graft of claim 17, wherein the scaffold comprises at least one of biodegradable magnesium or a biodegradable magnesium alloy.

19. The stent-graft of claim 1, wherein a thickness of the one or more struts is in a range of 200 μm to 1000 μm.

20. The stent-graft of claim 1, wherein an amplitude of the one or more struts is in a range of 2 mm to 6 mm.
